(19) United States
(12) Patent Application Publication
(10) Pub. No.: US 2014/0303383 A1
(43) Pub. Date: Oct. 9, 2014

(54) METHOD FOR PURIFYING LEVO-OXIRACETAM

(71) Applicants: Lei Ye, Yubei (CN); Zuyuan Rong, Yubei (CN)

(72) Inventors: Lei Ye, Yubei (CN); Zuyuan Rong, Yubei (CN)

(21) Appl. No.: 14/237,895

(22) PCT Filed: Apr. 23, 2012
(86) PCT No.: PCT/CN2012/074516

§ 371 Date: Apr. 10, 2014

(30) Foreign Application Priority Data
Aug. 11, 2011 (CN) 201110229931.5

(51) Int. Cl. C07D 207/273

(52) U.S. Cl. CPC C07D 207/273
USPC 548/544

(57) ABSTRACT

A method for purifying levo-oxiracetam is disclosed. Crude levo-oxiracetam is dissolved in water; an organic solvent is added dropwise into the solution until the solution just turns turbid; the solution is allowed to stand for 1 to 3 days at 0° C. to 18° C.; a colorless transparent crystal is precipitated; the solution is filtered and top-washed with cold water of 0° C. to 5° C.; and the product is dried in vacuum to obtain high-purity levo-oxiracetam.

### FIELD OF THE INVENTION

The present invention relates to a crystal purification method, in particular to the method for purifying (S)-4-hydroxy-2-oxo-1-pyrrolidine acetamide.

### BACKGROUND OF THE INVENTION

Oxiracetam (Olaxiracetam) is a nootropic drug which was first synthesized by Smithkline Beecham (Italia) and launched in Italy in 1987. levo-oxiracetam is a single enantiomer with a chemical name of (S)-4-Hydroxy-2-oxo-1-pyrrolidineacetamide (hereinafter referred to as “levo-oxiracetam).

Oxiracetam is capable of promoting the synthesis of phosphorylcholine and phosphoryl ethanol, promoting brain metabolism, and providing a stimulating effect to specific central nervous pathway through blood brain barrier to improve the ATP/ADP ratio of the brain and enhance the synthesis of brain protein and nucleic acid, so as to improve the memory and learning ability of mentally retarded patients, and the drug itself is not vascular active or causes any stimulation to the central nervous system, but this drug has a persistent promoting effect on learning and memory.

P.R.C. Pat. Nos. CN1513836, CN1948285 and CN101121688 have disclosed methods for synthesizing a racemate composed of two isomers, respectively: L-Oxiracetam and R-Oxiracetam. P.R.C. Pat. Nos. CN101367757 and CN101575309 have disclosed methods for preparing L-Oxiracetam. P.R.C. Pat. Nos. CN1424034, CN1555794, CN1562000 and CN101152175 have disclosed methods for preparing Oxiracetam injection agent, dispersible tablets, and lyophilized as well as a new formulation. International Pat. No. WO 93/06826 has discloses a method for improving the treatment effect with regard to intelligence by Oxiracetam. However, the purification process of levo-oxiracetam is relatively complicated or the purity of the product is relatively low, so that it is difficult to obtain high-purity levo-oxiracetam at a low cost by a simple manufacturing process.

### SUMMARY OF THE INVENTION

Therefore, it is a primary objective of the present invention to provide a method of purifying levo-oxiracetam in order to prepare levo-oxiracetam with low impurity (or high purity), and the method is simple and low-cost.

To achieve the aforementioned objective, the present invention provides a method of purifying levo-oxiracetam, comprising the following steps:

1. Dissolve crude levo-oxiracetam in water; drop an organic solvent into the solution and mix the solution thoroughly; allow the solution to stand still at 0° C. to 18° C. for 1 to 3 days; and precipitate the solution to obtain a colorless transparent crystal.

2. Filter and top-wash the colorless transparent crystal with cold water of 0° C. to 5° C.

3. Dry the product by vacuum to obtain high-purity levo-oxiracetam.

To thoroughly precipitate the levo-oxiracetam dissolved in water, the ratio of mass to volume (g/ml) of the crude levo-oxiracetam and water adopted in the step 1 is preferably 1:0.5˜2, and more preferably 1:0.75, and the temperature for the solution to stand still as described in the step 1 is preferably 5° C. to 15° C., and more preferably 9° C.

To further improve the purity of the final product of levo-oxiracetam, the organic solvent used in the step 1 is preferably ethanol, propanol or isopropanol.

To further improve the purity of the final product of levo-oxiracetam, and the temperature of the ice water set in the step 2 is preferably at 1° C. to 3° C. and more preferably 2° C., and the ratio of mass to volume (g/ml) of the precipitated crystal and ice water as described in the step 2 is preferably 1:1-2.

Preferably, the drying in vacuum as described in the step 3 takes place at a temperature of 26° C. to 28° C. for 4-5 hours.

Specifically, a method of purifying levo-oxiracetam comprises the following steps:

1. Dissolve crude levo-oxiracetam (with purity ≦89%) in water; add propanol dropwise into the solution and mix the solution thoroughly; allow the solution to stand still at 9° C. for 29˜30 hours; and add propanol dropwise into the solution, precipitate the solution to obtain a colorless transparent crystal, wherein the ratio of mass to volume (g/ml) of the crude levo-oxiracetam and water is 1:0.75.

2. Filter and top-wash the colorless transparent crystal with cold water, wherein the ratio of mass to volume (g/ml) of the precipitated crystal and ice water used in the step 1 is preferably 1:1-2, and the ice water is at 2° C.

3. Dry the product in vacuum at 26° C. to 28° C. for 4-5 hours to obtain high-purity levo-oxiracetam.

The present invention has the following advantages and effects: levo-oxiracetam has a high solubility, so that it will be dissolved quickly by water, and most people believe that it is infeasible to use water as a solvent to purify levo-oxiracetam. The inventor of the present invention also had the same thought at the beginning, and thus adopted organic solvents to purify levo-oxiracetam. The inventor of the present invention has conducted extensive experiments and studies different processes and manufacturing conditions, but the purity of the final product of the levo-oxiracetam is still unsatisfactory. In one of the experiments, the inventor of the present invention discovered that using water together with the organic solvent to purify crude levo-oxiracetam of 89% can improve the HPLC purity to 97.5%˜98.2%. In addition, this simple method has the features of mild control conditions, low production cost, free of pollution caused by organic solvents, eco-friendly, and applicable for large-scale industrial production.

### DESCRIPTION OF THE PREFERRED EMBODIMENTS

The aforementioned and other objectives and advantages of the present invention will become clearer in light of the following detailed description of an illustrative embodiment of this invention. It is intended that the embodiments disclosed herein are to be considered illustrative rather than restrictive.

### Preferred Embodiment 1

A method of purifying levo-oxiracetam, comprising the steps of: dissolving 1 g of crude levo-oxiracetam (purity of 89%) into 0.75 g of water; dropping propanol dropwise into the solution and then mixing the solution thoroughly; allowing the solution to stand still at 9° C. for 30 hours; precipitating the solution to obtain a colorless transparent crystal; filtering and top-washing the colorless transparent crystal with 2 ml of cold water at 2° C.; and drying the product in vacuum at 28° C. for 4.5 hours to obtain 0.5 g of a colorless crystal with HPLC purity of 98.2%.

More specifically, the aforementioned method of preparing crude levo-oxiracetam comprises the following steps:

(a) Put 28.50 g of glycinamide hydrochloride, 20.65 g of sodium bicarbonate and 200 ml of anhydrous ethanol into three reaction flasks respectively, and control the pH value to approximately 7.4, stir the solution to increase the temperature until a reflux occurs;

(b) Drop 39.08 g of S-4-chloro-3-hydroxybutyate after the reflux takes place for 2 hours. In the dropping process, 5 patches of the remaining of 20.65 g the sodium bicarbonate are added gradually, and the pH value is checked and controlled for each time when the alkaline is added, so as to ensure the pH value ≦8.5.

(c) After the dropping, (S)-4-chloro-3-hydroxybutyrate is refluxed for 24 hours until the HPLC testing product (S)-4-hydroxy-2-oxo-1-pyrrolidinyl acetamide content reaches 74%, and the crude (S)4-hydroxy-2-oxo-1-pyrrolidinyl acetamide is obtained after the heating, filtering and concentrating processes.

(d) The above-mentioned crude product is dissolved in 50 ml of water, and processed by 500 ml of 001x7 strong acidic styrene cation-exchange resin, and the product is collected. Use 201x7 strong alkaline styrene anion-exchange resin for neutralization and collect the resulted aqueous solution. The neutralization is determined to be finished when the pH value of the solution reaches 7.0±0.1.

### Preferred Embodiment 2

A method of purifying levo-oxiracetam, comprising the steps of: dissolving 5 Kg of crude levo-oxiracetam (purity of 89%) into 6 liters of water; dropping an organic solvent such as ethanol, propanol or isopropanol dropwise into the solution, and then mixing the solution thoroughly; allowing the solution to stand still at 12° C. for 29 hours; precipitating the solution to obtain a colorless transparent crystal; filtering and top-washing the colorless transparent crystal with 4 liters of cold water at 5° C.; and drying the product in vacuum at room temperature for 5 hours to obtain 2.5 Kg of a colorless crystal with HPLC purity of 98%.

### Preferred Embodiment 3

A method of purifying levo-oxiracetam, comprising the steps of: dissolving 1 Kg of crude levo-oxiracetam (purity of 89%) into 1.5 liters of water; adding an organic solvent THF into the solution and then mixing the solution thoroughly; allowing the solution to stand still at 6° C. for 30 hours; precipitating the solution to obtain a colorless transparent crystal; filtering and top-washing the colorless transparent crystal with 0.5 liter of cold water at 5° C.; and drying the product in vacuum at 28° C. for 4 hours to obtain 0.4 Kg of colorless crystal with HPLC purity of 97.8%.

While the invention has been described by means of specific embodiments, numerous modifications and variations could be made thereto by those skilled in the art without departing from the scope and spirit of the invention set forth in the claims.

What is claimed is:

1. A method of purifying levo-oxiracetam, comprising the steps of:
(1) dissolving crude levo-oxiracetam in water, dropping an organic solvent into the solution and then mixing the solution thoroughly, allowing the solution to stand still at 0° C. to 18° C. for 1 to 3 days, and precipitating the solution to obtain a colorless transparent crystal;
(2) filtering and top-washing the colorless transparent crystal with cold water from 0° C. to 5° C.;
(3) drying the product in vacuum to obtain high-purity levo-oxiracetam.

2. The method of purifying levo-oxiracetam according to claim 1, wherein the ratio of mass to volume (g/ml) of the crude levo-oxiracetam and water used in the step (1) is 1:0.5˜2, and the temperature set in the step (1) for the water to stand still is from 5° C. to 15° C.

3. The method of purifying levo-oxiracetam according to claim 2, wherein the ratio of mass to volume (g/ml) of the crude levo-oxiracetam and water used in the step (2) is 1:075, and the temperature set in the step (1) for the water to stand still is 9° C.

4. The method of purifying levo-oxiracetam according to any one of claims 1 to 3, wherein the organic solvent is one selected from the group consisting of ethanol, propanol, and isopropanol.

5. The method of purifying levo-oxiracetam according to any one of claims 1 to 3, wherein the ice water used in the step (2) has a temperature of 1° C. to 3° C., and the ratio of mass to volume (g/ml) of the precipitated crystal and ice water as described in the step (1) is preferably 1:1˜2.

6. The method of purifying levo-oxiracetam according to claim 5, wherein the ice water used in the step (2) has a temperature of 2° C.

7. The method of purifying levo-oxiracetam according to any one of claims 1 to 6, wherein the drying in vacuum as described in the step 3 takes place at a temperature of 26° C. to 28° C. for 4-5 hours.

8. The method of purifying levo-oxiracetam according to claim 1, comprising the steps of:
(1) dissolving crude levo-oxiracetam (purity 89%) in water, dropping propanol dropwise into the solution and then mixing the solution thoroughly, allowing the solution to stand still at 9° C. for 29˜30 hours, and precipitating the solution to obtain a colorless transparent crystal, wherein the ratio of mass to volume (g/ml) of the crude levo-oxiracetam and water is 1:0.75;
(2) filtering and top-washing the colorless transparent crystal with cold water from 0° C. to 5° C., wherein the ratio of mass to volume (g/ml) of the precipitated crystal and ice water is preferably 1:1˜2, and the ice water is at 2° C.; and
(3) drying the product in vacuum at 26° C. to 28° C. for 4˜5 hours to obtain high-purity levo-oxiracetam.
